(19) United States
(12) Plant Patent Application Publication
(10) Pub. No.: US 2019/0269056 P1
(43) Pub. Date: Aug. 29, 2019

(54) Hibiscus plant named 'Ruthib1'

(50) ;

(71) Applicant: University of Georgia Research Foundation, Inc., Athens, GA (US)

(72) Inventor: John M. Ruter, Bishop, GA (US)

(73) Assignee: University of Georgia Research Foundation, Inc., Athens, GA (US)

(21) Appl. No.: 15/932,408

(22) Filed: Feb. 26, 2018

(51) Int. Cl. A01H 6/60

(52) U.S. Cl. PLT/257

(57) ABSTRACT

A new Hibiscus plant named ‘RutHib1’ is characterized by a combination of good form, medium vigor, predominately purple foliage, white flowers with a red eye, and good tolerance to Alternaria, Cercospora, and Phytophthora pathogens.

Genus and species: Hibiscus moscheutos.

Variety denomination: The new Hibiscus moscheutos claimed is of the cultivar denominated ‘RutHib1.’

### BACKGROUND OF THE INVENTION

The present invention relates to a new and distinct cultivar Hibiscus moscheutos, botanically known as Hibiscus moscheutos ‘RutHib1.’

The new Hibiscus moscheutos ‘RutHib1’ is a product of a planned breeding program conducted by the Inventor in Watkinsville, Ga. The objective of the Hibiscus breeding program is to create new plant cultivars with ornamental leaf distinctions, abundant flowers, and tolerance to pathogens.

The new Hibiscus moscheutos ‘RutHib1’ is a product of Hibiscus moscheutos ‘13-19’ (Inventor controlled cross, not patented) x ‘Crown Jewels’ (U.S. Plant Pat. No. 11,857). The female parent is Hibiscus moscheutos ‘13-19’. The cross was made in 2011. ‘RutHib1’ has been evaluated through trials in Watkinsville, Ga. from 2012 and the plant ‘RutHib1’ was selected in 2013.

Asexual reproduction of the new Hibiscus moscheutos ‘RutHib1’ was by vegetative terminal cuttings in a controlled environment in Watkinsville, Ga. since 2013. Observations of the resulting ‘RutHib1’ progeny has shown that the unique features of this new Hibiscus moscheutos ‘RutHib1’ are stable and reproduced true to type in successive generations.

### SUMMARY OF THE INVENTION

The cultivar ‘RutHib1’ has not been observed under all possible environmental conditions. The phenotype may vary somewhat with variations in environment and cultural practices such as temperature and light intensity without, however, any variance in genotype.

The following represents the distinguishing characteristics of the new Hibiscus moscheutos cultivar named ‘RutHib1’. In combination, these traits set ‘RutHib1’ apart from all other existing varieties of Hibiscus known to the inventor.
1. Good Form.
2. Medium vigor.
3. Predominately purple foliage.
4. White flowers with a red eye.
5. Good tolerance to Alternaria, Cercospora, and Phytophthora pathogens.

### COMPARISON TO PARENT PLANTS

RutHib1’ is more upright in growth habit than ‘Crown Jewels’. Hibiscus ‘13-19’ is taller than ‘RutHib1’; has a more open growth habit and less glossy leaves than ‘RutHib1’.

### BRIEF DESCRIPTION OF THE FIGURES

The accompanying colored photographic illustrations show the overall appearance and distinct characteristics of the new cultivar of Hibiscus moscheutos ‘RutHib1’. The colors in the photographs are as close as possible with the photographic and printing technology utilized.

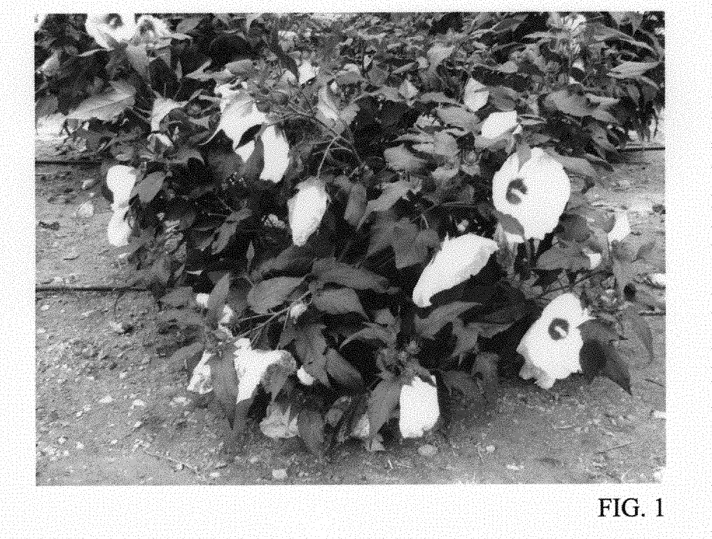

The photograph labeled FIG. 1 depicts a five year old ‘RutHib1’ plant.

### DETAILED BOTANICAL DESCRIPTION

The following is a detailed description of the Hibiscus moscheutos cultivar named ‘RutHib1’. Data was collected at a farm in Watkinsville, Ga. from five year old plants grown outdoors and planted in the ground.

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society (R.H.S.), London, England.
Habit: Upright.
Size of plant:
   A. Height (cm).—74.93.
   B. Width (cm).—152.40.
Rooting: The new plant typically initiates roots in fourteen to twenty-one days under favorable conditions.
Stem:
   A. Color.—Greyed-Brown 199-A.
   B. Length.—88.90 cm.
   C. Diameter.—1.3 cm (mid stem).
   D. Pubescence.—None.
   E. Shape.—Round.
   F. Odor (of bruised stem).—Grassy.
   G. Internode length.—3.2 cm-5.7 cm.
   H. Average number of stems/branches.—50-70.
Leaf:
   A. Color (R.H.S.).—1. Upper: Predominately greyed-purple 187-A. 2. Lower: Green 137-C.
   B. Mature size (l×w).—19.5 cm×16.8 cm.
   C. Apex.—Acuminate.
   D. Base.—Cordate.
   E. Margin.—Erose.
   F. Shape.—Falcate.
   G. Lobes.—Three lobes, lobing is shallow.
   H. Pubescence.—None.
   I. Arrangement on stem.—Alternate.
   J. Venation.—Reticulate.
   K. Texture.—Glabrous (both surfaces of leaf).
Petiole:
   A. Length.—5.7 cm-10 cm.
   B. Shape.—Oval.
   C. Color (R.H.S.).—Greyed orange 165-A.
   D. Pubescence.—None.
   E. Diameter (mm).—3.3 mm.
Flower:
   A. Inflorescence.—1. Flowers per stem 13-63.
   B. Individual flower.—1. Axillary, terminal Axillary; terminal clusters. 2. Symmetry Actinomorphic. 3. Petals. a. Size (L×W) 10.7 cm×14.3 cm. b. Shape Obdeltoid. c. Apex Rounded. d. Base Orbicular. e. Margin Entire. f. Color at peak of bloom. i. Upper petal surface White N155-B; Veins — Red 55-B. ii. Lower petal surface White N155-B. iii. Eye Zone Red 53-A. g. Number of petals 5. h. Flowers are single. 4. Pedicels. a. Color (R.H.S.) Yellow-green 144-A. b. Pubescence None. c. Length 9.0 cm-9.8 cm. 5. Sepals. a. Number 5. b. Size (L×W) 4.5 cm×2.3 cm. c. Shape Ovate lanceolate. d. Pubescence Sparsely lanuginous. e. Color (R.H.S.) Yellow-green 144-A. f Texture Both surfaces slightly hairy which provides texture. 6. Stamens. a. Number 135. b. Size (L×W) 0.4 cm×<0.1 cm. c. Color (R.H.S.) 158-A. d. Pollen Color 158-B. e. Pubescence None. 7. Pistils. a. Number 1 pistil with 5 stigmas. b. Size (L×W) 5.7 cm×0.6 cm. c. Color of Style (R.H.S.) Yellow-white 158-B. d. Color of Stigma (R.H.S.) Yellow-white 158-A. 8. Bracts. a. Number 12. b. Size 2.85 cm (L); 0.1 cm-0.3 cm (tapered width). c. Color (R.H.S.) Green 143-A. C. Fruit: None observed to date. D. Seed: None observed to date. E. Bloom time: In Watkinsville, Ga., plants typically begin blooming in mid-May and continue blooming until early July. F. Fragrance: No apparent fragrance.
Winter hardiness: The plant has been observed in Watkinsville, Ga., U.S. hardiness zone 8a; and in West Grove, Pa., U.S. hardiness zone 6b.

What is claimed is:

1. A new and distinct cultivar of the Hibiscus plant named ‘RutHib1’ as illustrated and described herein.
